(19) United States
(12) Patent Application Publication
(10) Pub. No.: US 2012/0265222 A1
(43) Pub. Date: Oct. 18, 2012

(54) SYSTEM AND METHOD FOR FORMING A KNOT

(75) Inventors: Udi Gordin, Yuvalim (IL); Gilad Heftman, Kibutz Ein Gev (IL); Yoav Mintz, Jerusalem (IL)

(73) Assignee: VIRTUAL PORTS LTD., Caesarea (IL)

(21) Appl. No.: 13/499,829

(22) PCT Filed: Oct. 3, 2010
(86) PCT No.: PCT/IL10/00799

§ 371 Date: Jun. 26, 2012

Related U.S. Application Data
(60) Provisional application No. 61/247,582, filed on Oct. 1, 2009.

(51) Int. Cl. A61B 17/04

(52) U.S. Cl. 606/148

(57) ABSTRACT

The present invention provides a suturing device for internally forming a knot in a suture within a body cavity, during surgeries, wherein said device comprising a flexible pre-shaped helical lumen having two opposite ends, said lumen comprising n loops; n is a real number higher than or equal to 1; said helical lumen is adapted to accommodate at least a portion of said suture, such that said knot is obtained by threading by at least one end of said suture through at least one of said loops.

### FIELD OF THE INVENTION

The present invention relates to a device simplifying the tying of suture knots, which will prove especially useful in laparoscopic surgery and minimal invasive surgeries (MIS).

### BACKGROUND OF THE INVENTION

In the prior art a variety of systems for aiding in the tying of suture knots have been presented.

Amongst the techniques used are the tying of traditional knots by various means, or avoiding the tying of, knots by means of fusing suture sections e.g. by heat or ultrasound, or anchoring suture ends in bone. The instant invention proposes a novel knot-tying system and method, thus avoiding the complications inherent in the latter techniques.

Amongst the knot tying techniques, one common method is to use a ‘taut line hitch’ in the suture ends e.g. outside the body, and then to pull this knot along the suture to its eventual location. However as is known to those skilled in the art a sawing motion is required to pull the knot down the line which often abrades or cuts the delicate bodily tissues in the environs. Similarly, a knot can be formed in the suture remote from its eventual location, and run down to its eventual location, and doing the same with a second and further knot, running down a series of knots as necessary. The knot is formed by laying one or more knots one upon another and tightening the suture so as to lock the strands of suture together. For example, U.S. Pat. No. 5,084,058 provides a suture knot holder, rundown tool and cutting system for running one or more knots formed in two lengths of suture extending from a surgical site down to the surgical site so as to form a knot at the surgical site and for blindly cutting the suture ends adjacent the knot. The system comprises a support mechanism for releasably supporting a plurality of knots in a predetermined arrangement adjacent a surgical site, and a tool for removing knots from the support mechanism, running the knots down the lengths of the suture to the surgical site, and blindly cutting the suture ends at substantially equal lengths every time adjacent a knot formed by the knots at the surgical site.

However it will be appreciated that this method also requires the pushing of knots down a suture line, a laborious procedure rife with peril due to the delicacy of tissues surrounding the suture and relatively great lengths of several tens of centimeters often required to push the knots.

U.S. Pat. No. 6,296,648 provides a sewing appliance for minimally invasive surgery comprising a shaft whose distal end is provided with at least one plane support that can be swung out laterally. The device is inserted into the laparoscopic cavity with its ‘wings’ folded back. Once it has penetrated e.g. the abdomen wall, the ‘wings’ unfold to be perpendicular to the rod, and when withdrawn the wings are folded forwards to again be parallel to the rod. However as mentioned in the patent draft this device is intended to deliver suture thread and tying device to the desired locations, but does not itself constitute a suture tying device.

U.S. Pat. No. 5,480,406 contemplates a method for using an endoscopic suturing apparatus to conveniently place various knots in suture such as a square knot, a modified fisherman's knot, a securing knot, an alternative square knot and a surgeon's knot. The knots are formed variously by placing the suture between the jaws or around one jaw and passing a needle (through which the suture end is threaded) from one jaw to the other to be able to draw the suture through loops formed in the suture. It will be appreciated however that the operation of passing the needle from jaw to jaw may be difficult as both jaws will grasp the needle with equal tension, or if one grasps with greater tension it will be impossible to pass the needle to the other jaw. Furthermore the various knot-tying procedures outlined will require some minimum of space that may or may not be available in a given surgical situation.

Hence, an improved system or device and method for tying knots in suture is still a long felt need.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which

FIGS. 1a-1m illustrate the suture knot tying method according to one embodiment of the present invention;

FIGS. 2a, 2b present further embodiments of the coil according to the present invention;

FIGS. 3a-3d illustrate a further embodiments of the coil according to the present invention;

FIGS. 4a-4d illustrate another embodiment of the present invention in which an at least partially coiled suture is used.

### SUMMARY OF THE INVENTION

The present invention comprises a system and method for aiding the tying of knots in sutures. In one embodiment a coil is provided that is adapted to be threaded onto a suture, forming a helical structure. One end of the suture is threaded through the far end of the helix, thereby forming knots in the suture which can be pulled taut to form a knot. In another embodiment the suture itself is formed in a helical fashion.

It is one object of the present invention to provide a suturing device for internally forming a knot in a suture within a body cavity, during surgeries, wherein said device comprising a flexible pre-shaped helical lumen having two opposite ends, said lumen comprising n loops; n is a real number higher than or equal to 1; said helical lumen is adapted to accommodate at least a portion of said suture, such that said knot is obtained by threading by at least one end of said suture through at least one of said loops.

It is another object of the present invention to provide the suturing device as defined above, wherein said lumen is made of materials selected from a group consisting of shape memory materials, polymers, metals, plastics, transparent materials, biocompatible materials, bioabsorbable materials, and drug-treated materials.

It is another object of the present invention to provide the suturing device as defined above, wherein said device additionally comprising a grabable portion adapted to be pulled such that said flexible pre-shaped helical lumen is transformed into a substantially straight lumen.

It is another object of the present invention to provide the suturing device as defined above, additionally comprising at least one drag coupled to one end of said lumen so as to distinguish between the two ends of the same.

It is another object of the present invention to provide the suturing device as defined above, wherein one end of said lumen additionally comprises indicating means adapted to indicate a proper threading of said at least one of said suture is obtained.

It is another object of the present invention to provide the suturing device as defined above, wherein said lumen comprises at least one groove and/or at least one opening on the outer surface of the same; said groove and/or said opening is adapted to facilitate threading of said suture through said lumen.

It is another object of the present invention to provide the suturing device as defined above, wherein the cross sectional area of said lumen is selected from a group consisting of circular, polynomial rectangular or any combination thereof.

It is one object of the present invention to provide a method for internally forming a knot made of at least one loop in a suture within a body cavity, during surgeries. The method comprising steps selected from:
a. obtaining a suturing device, said suture device comprising a flexible pre-shaped helical lumen having two opposite ends, said lumen comprising n loops; n is a real number higher than or equal to 1;
b. threading a first end of said suture through said first end of said pre-shaped helical lumen; thereby accommodating at least a portion of said suture within said helical lumen;
c. passing at least one end of said suture through y loops of said helical lumen from the opposite end of said lumen; y is an integer less than or equals to n; and,
d. pulling said passed end of said suture, thereby providing said knot.

It is another object of the present invention to provide the method as defined above, additionally comprising step of repeating said step of passing at least one end of said suture through at least one loop of said helical lumen from the opposite end of said lumen k times, where k is an integer higher than 1.

It is another object of the present invention to provide the method as defined above, additionally comprising step un threading said suture from said pre-shaped helical lumen by pulling out said pre-shaped helical lumen.

It is another object of the present invention to provide the method as defined above, additionally comprising step of forming k knots.

It is another object of the present invention to provide the method as defined above, additionally comprising step of selecting the materials from which said lumen is made of from a group consisting of shape memory materials, polymers, metals, transparent materials, biocompatible materials, bioabsorbable materials, and drug-treated materials.

It is another object of the present invention to provide the method as defined above, additionally comprising step of grasping and pulling a grabable portion (i.e., the drag) such that said flexible pre-shaped helical lumen is transformed into a substantially straight lumen.

It is another object of the present invention to provide the method as defined above, additionally comprising step of forming said lumen with at least one drag coupled to one end of said lumen so as to distinguish between the two ends of the same.

It is another object of the present invention to provide the method as defined above, additionally comprising step of providing said lumen with at least one groove and/or at least one opening on the outer surface of the same for facilitating said step of threading of said suture through said lumen.

It is another object of the present invention to provide the method as defined above, additionally comprising step of selecting the cross sectional area of said lumen from a group consisting of circular, polynomial rectangular or any combination thereof

It is another object of the present invention to provide the method as defined above, additionally comprising step of indicating proper threading of said at least one of said suture is obtained trough said lumen.

It is another object of the present invention to provide a suture for internally forming a knot in a suture within a body cavity, during surgeries, wherein said suture comprises at least one coiled section (helicoil) characterized by a predetermined number of loops, and adapted for being threaded by one end of said suture, such that said knot is provided.

It is another object of the present invention to provide the suture as defined above, wherein said at least one coiled section (helicoil) is provided by means selected from extrusion, thermal treatment, plastic deformation, gluing or any combination thereof

It is another object of the present invention to provide the suture as defined above, wherein said suture is made of material selected from a group consisting of medical sutures, shape memory materials, nitinol, polymers, metals, transparent materials, biocompatible materials, bioabsorbable materials, non-bioabsorbable materials, mono/multi Filament and drug-treated materials.

It is another object of the present invention to provide the suture as defined above, wherein the diameter of said coiled section (helicoil) is in the range of about 1 mm to about 50 mm.

It is another object of the present invention to provide the suture as defined above, wherein the helicity (i.e., pitch) of said coiled section (helicoil) is in the range of about 0 mm to about 50 mm.

It is another object of the present invention to provide the suture as defined above, wherein said coiled section (helicoil) is characterized by a varying diameter and helicity (i.e., pitch).

It is another object of the present invention to provide the suture as defined above, additionally comprising a second suture; wherein said second suture is characterized by at least one selected from a group consisting of mechanical properties, chemical properties or any combination thereof which are substantially different than said suture.

It is another object of the present invention to provide the suture as defined above, additionally comprising coupling means 32 adapted to couple said suture and said second suture; wherein said coupling is provided via mechanical means, chemical means, physical means or any combination thereof.

It is another object of the present invention to provide the suture as defined above, wherein said coupling is provided by at least one selected from a group consisting of hooks and loops, cramping, ratchet, glue or any combination thereof.

It is another object of the present invention to provide the suture as defined above, additionally comprising balancing element 33 adapted to balance said suture.

It is another object of the present invention to provide a method for internally forming a knot made of at least one loop in a suture within a body cavity, during surgeries. The method comprising steps selected from:
a. providing a suture having at least one coiled section (helicoil); said coiled section comprises preset n loops; n is a real number higher or equal to 1;
b. passing at least one end of said suture through y loop of said coiled; y is an integer less than or equals to n; and,
c. pulling said passed end of said suture, thereby forming a knot therein.

It is another object of the present invention to provide the method as defined above, additionally comprising step of forming said suture from material selected from a group consisting of medical sutures, shape memory materials, nitinol, polymers, metals, transparent materials, biocompatible materials, bioabsorbable materials, non-bioabsorbable materials, mono/multi Filament and drug-treated materials.

It is another object of the present invention to provide the method as defined above, additionally comprising step of providing the diameter of said helicoil is in the preferred range of about 1 mm to about 50 mm.

It is another object of the present invention to provide the method as defined above, additionally comprising step of forming said at least one coiled section (helicoil) by means selected from extrusion, thermal treatment, plastic deformation, gluing or any combination thereof.

It is another object of the present invention to provide the method as defined above, additionally comprising step of providing the helicity (i.e., pitch) of said coiled section (helicoil) is in the range of about 0 mm to about 50 mm.

It is another object of the present invention to provide the method as defined above, additionally comprising step of providing a second suture; wherein said second suture is characterized by at least one selected from a group consisting of mechanical properties, chemical properties or any combination thereof which are substantially different than said suture.

It is another object of the present invention to provide the method as defined above, additionally comprising step of coupling said suture and said second suture.

It is another object of the present invention to provide the method as defined above, wherein comprising step of coupling is provided via mechanical means, chemical means, physical means or any combination thereof.

It is another object of the present invention to provide the method as defined above, wherein comprising step of coupling is provided by at least one selected from a group consisting of hooks and loops, cramping, ratchet, glue or any combination thereof

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for tying suture knots.

In the following detailed description, numerous specific details are set forth in order to provide a thorough understanding of embodiments of the present invention. However, those skilled in the art will understand that such embodiments may be practiced without these specific details. Reference throughout this specification to “one embodiment” or “an embodiment” means that a particular feature, structure, or characteristic described in connection with the embodiment is included in at least one embodiment of the invention.

The term ‘suture’ hereinafter refers to a thread or thread-like object, especially as used in surgical procedures to tie, sew, or thread together two or more locations in or on the body. Stitches, for example, are composed of suture material. The material itself can either be biodegradable or not, but is always biocompatible.

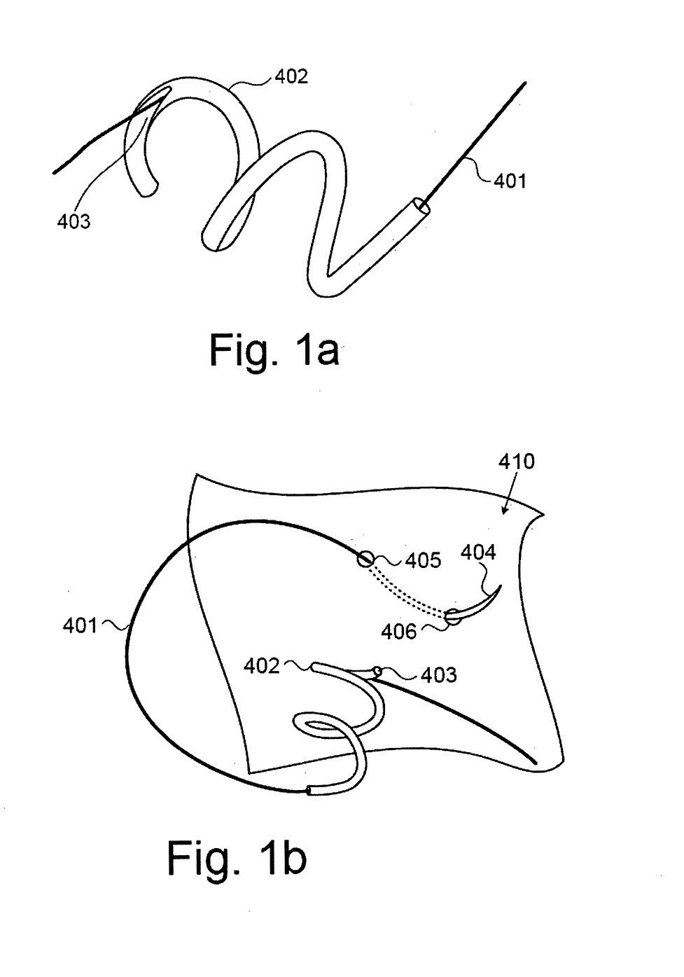

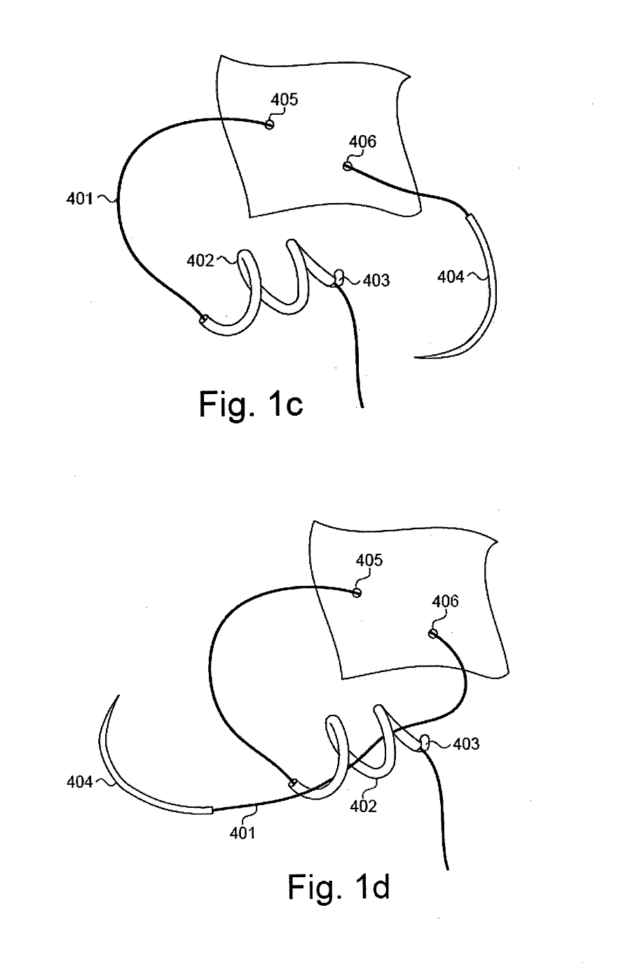

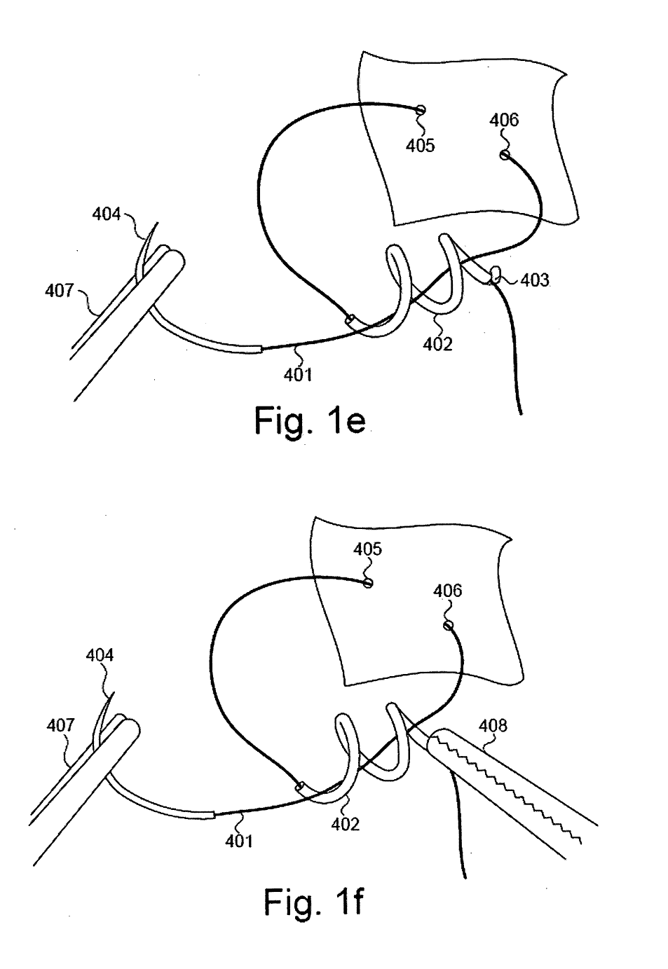

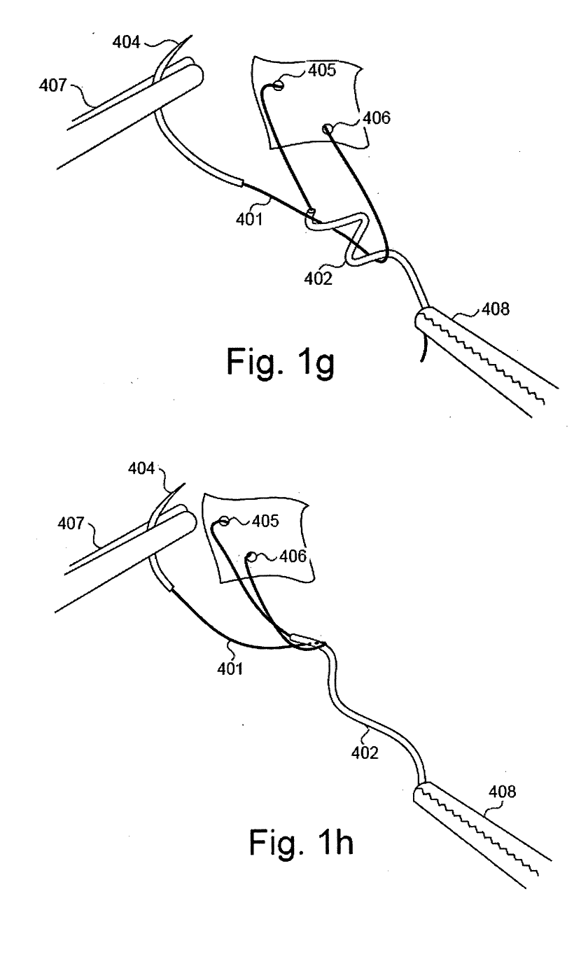

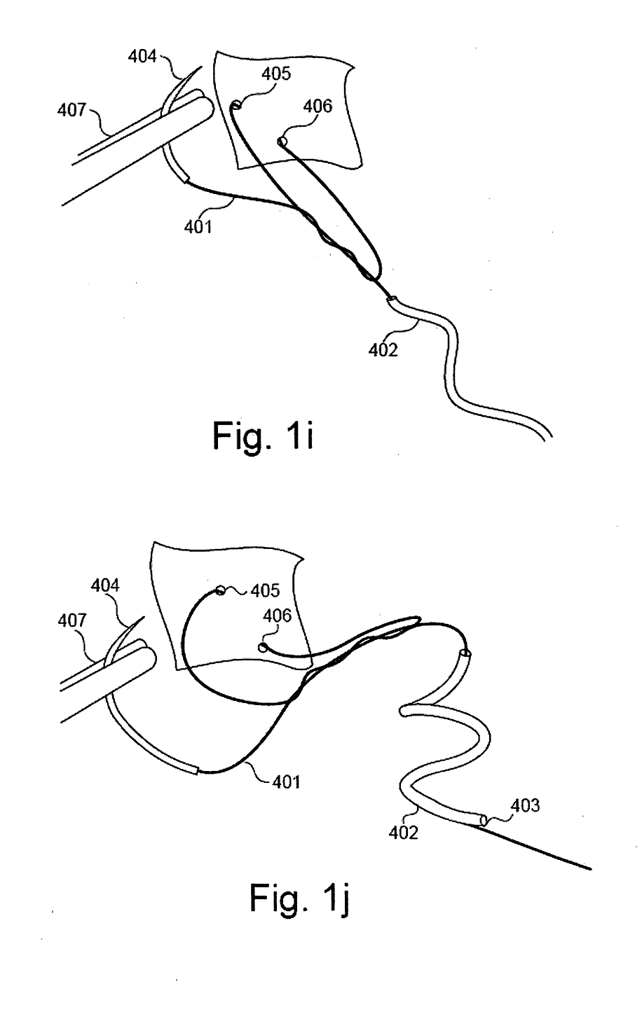

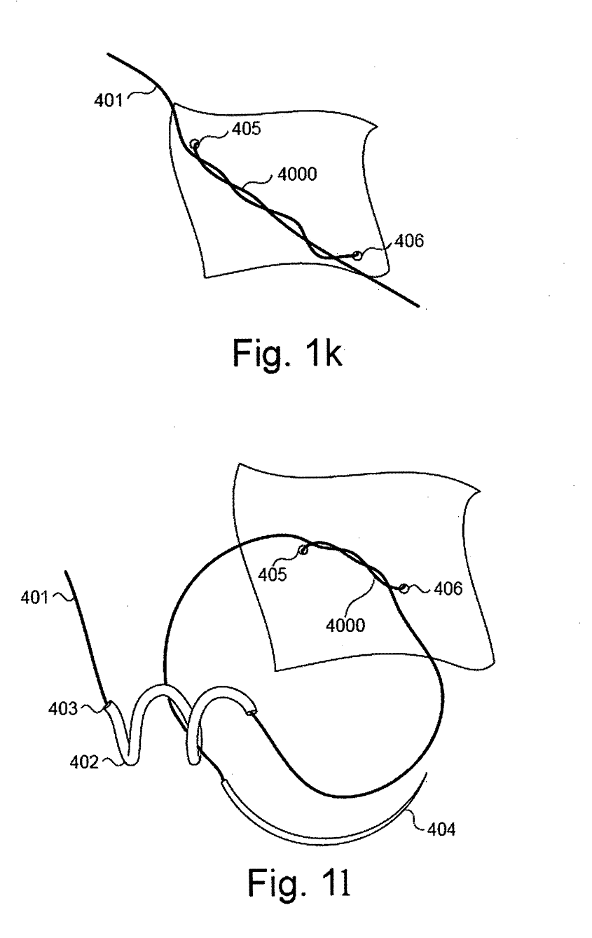

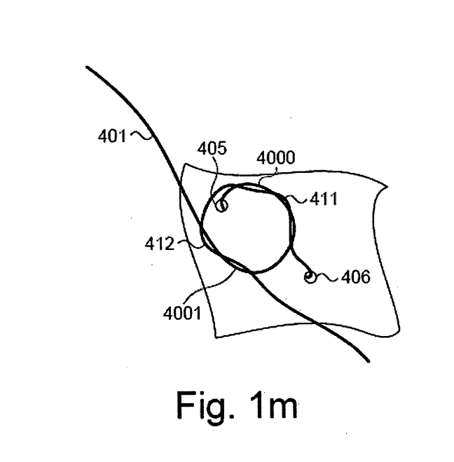

The term “knot” refers hereinafter to the formation of at least one tie made by threading one end of the suture through Y loops (see for example FIG. 1d for a formation of one knot 4000 made by threading of a suture 401 through 2 loops; and FIG. 11, 1m for the formation of a second knot 4001 made by threading suture 401 through one loop).

The term ‘plurality’ refers hereinafter to any positive integer e.g, 1,2, et cetera.

The term ‘surgery’ refers hereinafter to any type of surgery—both minimal invasive surgeries and open surgeries.

The term ‘forceps’ refers hereinafter to an instrument used to hold firmly or remove (i.e. pincers or tongs). Forceps are a handheld, hinged instrument used for grasping and holding objects. Another common term is a ‘needle holder’ which is used for grasping a suturing needle.

According to a preferred embodiment of the present invention, a hollow polymeric helical tube is threaded onto a suture. This helix forms a ‘tunnel’ through which the end of the suture can be threaded. Said threading of said suture is then utilized to form a knot, as will be described in the following.

FIGS. 1a-1m illustrate the first suturing device as provided by the present invention.

The first embodiment provides a suturing device for internally forming a knot in a suture within a body cavity. It is emphasized that the knot is formed during surgeries and especially in minimal invasive surgeries. The device, as will be described hereinafter, comprises a flexible pre-shaped helical lumen having two opposite ends. The lumen comprising n loops where n is a real number higher than or equal to 1.

Furthermore, the helical lumen is adapted to accommodate at least a portion of the suture, such that a knot is obtained by threading by at least one end of said suture through at least one of the loops.

In FIG. 1a one sees the flexible pre-shaped helical lumen 402 threaded onto a suture 401. As is evident from the figure, the helix is rigid enough that it imparts a helical shape to the suture. However, it is not so rigid that it cannot be deformed under moderate force.

One end of the helical lumen 403 is not fully closed and is formulated as a drag. It allows the used to distinguish the two ends of the helix at a glance. This will prove of importance during the tying of knots in the suture.

Yet more said end/drag 403 enables (in a later point) to enable grasping and pulling of the helical lumen via the forceps substantially without applying pressure on the suture itself.

It should be pointed out that in this specific example (FIG. 1a) the drag is an ‘open end’ of the helical lumen; however, it can be produced as an independent part which is coupled to the helical lumen.

Alternatively the drag can be a bulge on the outer surface of the lumen.

As described above, the lumen comprises n loops where n is a real number higher than or equal to 1. In the figure n is 2.

FIG. 1b illustrates the flexible pre-shaped helical lumen 402 threaded on the suture 401.

The suture has been threaded, using needle 404, through a piece of material 410 that is intended to represent human tissue. Thus, the suture enters at location 405, travels inside material 410 (in practice material 410 is the tissue), and exits at location 406.

In FIG. 1c the needle 404 has been pulled entirely out of the material 410.

In FIG. 1d the needle 404 has been pulled through the flexible pre-shaped helical lumen 402. In this particular helical lumen example, the suture is threaded through two loops. Said threading will eventually result in a knot with two loops within said loop.

Obviously the helix can be produced with an arbitrary number of loops, enabling knots of an arbitrary number of loops to be tied.

At this point we note that topologically speaking a knot has already been formed. It only remains to remove the helical lumen and pull the ends of the suture tight to close the knot securely.

In FIG. 1e laparoscopic forceps 407 are used to grasp needle 404.

In FIG. 1f a second pair of laparoscopic forceps 408 are used to grasp the end of the helical lumen 403 to prepare for pulling helical lumen out of the knot.

In FIG. 1g the first forceps 407 are used to pull the needle (and suture tied to it). The second forceps 408 pull the flexible pre-shaped helical lumen away from the knot location (by pulling the helical lumen's end 403. It is emphasized that while the lumen is characterized by mechanical properties enabling to maintain in a helical-shape, once the lumen is pulled, it shape is transformed to a substantially linear one so as to be eventually pulled out.

In FIG. 1h the first forceps 407 continue to pull the needle (and suture tied to it),.while the second forceps 408 continue to pull the helical lumen's end 403.

In FIG. 1i the first forceps 407 continue to pull the needle (and suture tied to it), causing the knot to tighten. The helical lumen is finally removed.

In FIG. 1j the first forceps 407 continue to hold the needle (and suture tied to it). The untied knot is ready for tightening. The helical lumen is ready to form additional knots, if needed. Alternatively, it can be pulled out of the suture.

In FIG. 1k the knot 4000 has been tightened. The knot is obtained while the helical lumen is extracted.

In FIG. 1l the knot 4000 is shown as well as the needle 404 and helical lumen 402. The needle has been passed through only one loop a second time, in order to add a second knot. This time through one loop.

In FIG. 1m the second knot 4001 has been pulled closer to the first knot 4000.

Additional knots can be added as many as needed.

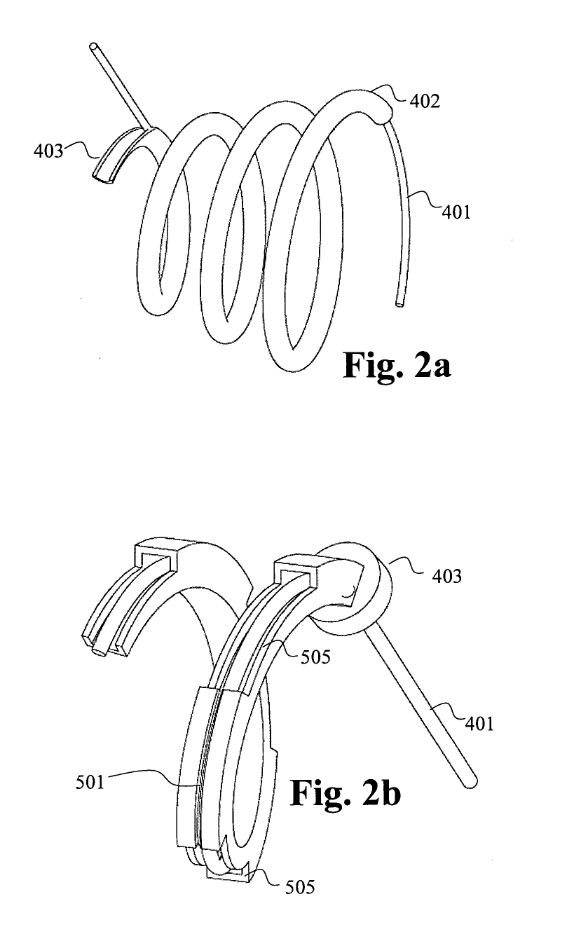

In FIG. 2a another embodiment of the helical lumen 402 can be seen. This helical lumen has been threaded on a suture 401, and as before one end 403 is not entirely closed, allowing it to be grasped and pulled along the suture. The diameter of the loops may vary to ease the insertion of the needle between the loops. According to one embodiment of the present invention, said end can be colored such that a user would easily recognize it.

In FIG. 2b an alternative embodiment is shown. The helical lumen is formed by polygonal cross section tube having openings so the suture is forced to be attached to the lumen in several locations only.

Longitudinal grooves 501 may be added to allow insertion (i.e., threading) of the suture to the center of the lumen.

At lest one Opening 505 may be added to ease the threading of the suture into the lumen.

The tip (i.e, the drag) 403 which is using to pull the helical lumen may protrude radially.

As explained, the grooves or the openings serve a serve important role—to ease the threading of the suture into the lumen. Since it is difficult to thread the suture into a lumen; and even more into a helical lumen, it would be advantageous to have said grooves or said openings which facilitated said threading.

Yet more, the more loops contained within said lumen the friction between the suture and the lumen will be obtained.

By furnishing the lumen with a plurality of said grooves 501 or/and openings 505 insertion of the suture directly into the lumen (without the need for threading the suture along the entire length of the lumen) is provided.

As can be seen from the figure, the cross sectional area of the lumen can be any of the following—circular, polynomial, polygon, rectangular or any combination thereof.

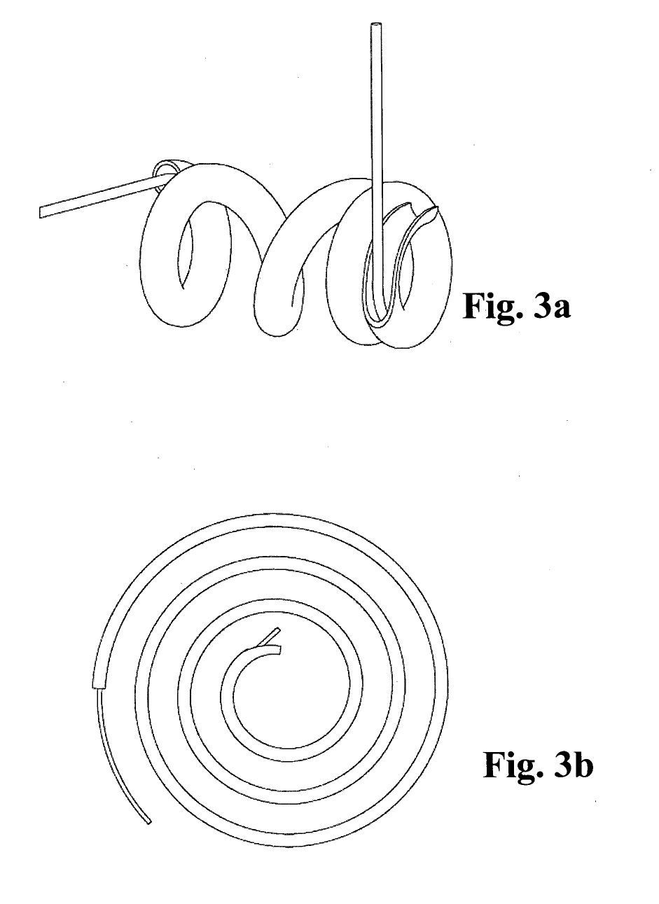

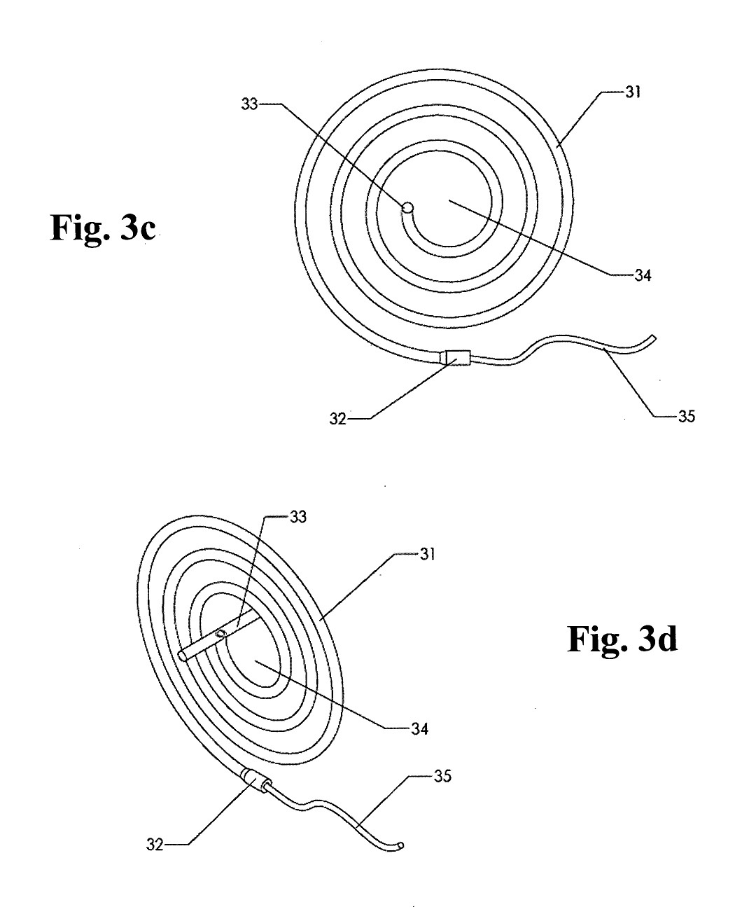

In FIG. 3a an alternative embodiment of the helical lumen is shown where the pitch of the helical lumen has been decreased allowing insertion of the needle between the oops easily. According to one embodiment of the present invention, the helical lumen can be characterized by a varying pitch.

In FIG. 3b still another embodiment of the helical lumen is showing two dimensions spiral. In this embodiment the pitch equals zero.

In this configuration the needle may be inserted in the center of the spiral to use all the loops or between to radially loops to use less loops.

According to another embodiment of the present invention, the lumen is made of materials selected from a group consisting of shape memory materials, polymers, metals, transparent materials, biocompatible materials, bioabsorbable materials, and drug-treated materials.

According to another embodiment of the present invention, the suturing device additionally comprising a grabable portion adapted to be pulled such that said flexible pre-shaped helical lumen is transformed into a substantially straight lumen.

According to another embodiment of the present invention, one end of said lumen additionally comprises indicating means adapted to indicate a proper threading of said at least one of said suture is obtained.

Reference is now made to FIGS. 3C-3D which provide a side view and an isometric view respectfully of another embodiment of the present invention.

According to this embodiment the suture 35 is coupled to one end of a pre-shaped spiral spring 31 (preferable the external end), and in fact is considered as a direct continuation of the same.

According to said embodiment, said pre-shaped spiral spring 31 is full and is not hollow. According to another embodiment said pre-shaped spiral spring 31 is in fact a suture.

It is emphasized that suture 35 may be a standard, off the shelf suture, and said pre-shaped spiral spring 31 is constructed both form materials and in shape so as to enable stretching forces as accepted in laparoscopic surgeries (namely up to 2 Kg).

According to another embodiment of the present invention the pre-shaped spiral spring 31 is constructed both form materials and in shape so as to enable the stretching of the same from its pre designed shape to a straight and linear shape (as is seen in FIG. 1h).

According to this embodiment the needle is threaded through center 34 (which is the center of the pre-shaped spiral spring 31) in order to obtain n loops.

According to this embodiment the internal end of the pre-shaped spiral spring 31 comprises an element 33 which can be used for (a) tightening and closing the knot securely; and/or (b) balancing element for the pre-shaped spiral spring 31.

Element 33 may be positioned at a varying angles with respect to the spiral spring 31 and may be shaped at any pre-desired shape.

Element 33 balances the pre-shaped spiral spring 31 by e.g., being reversibly coupled to the tissue.

In FIG. 3c element 33 is a linear rod which is positioned perpendicularly to the spiral spring 31; however, said element may take any shape and any position with respect to the spiral spring 31.

It should be emphasized that element 33 balances the spiral spring 31 by protruding in two directions out of the spiral spring 31 plane.

According to one embodiment the external end of the spiral spring 31 comprises coupling element 32 which is adapted to enable coupling of the suture 35 to the spiral spring 31.

The coupling can be obtained by mechanical, physical chemical or any combination thereof.

According to a specific embodiment of the present invention coupling is obtained magnetically, mechanical means (e.g., hooks and loops, cramping of the coupling means 32 onto the suture, ratchet), via adhesive means (e.g., glue) or any combination thereof.

According to another specific embodiment of the present invention in the center of the coupling element 32, a bore is provided which enables the insertion of suture 35 into the same.

According to this embodiment, the pre-shaped spiral spring 31 is symmetrical and hence the needle (and hence the suture 35) can be threaded from both sides of the same.

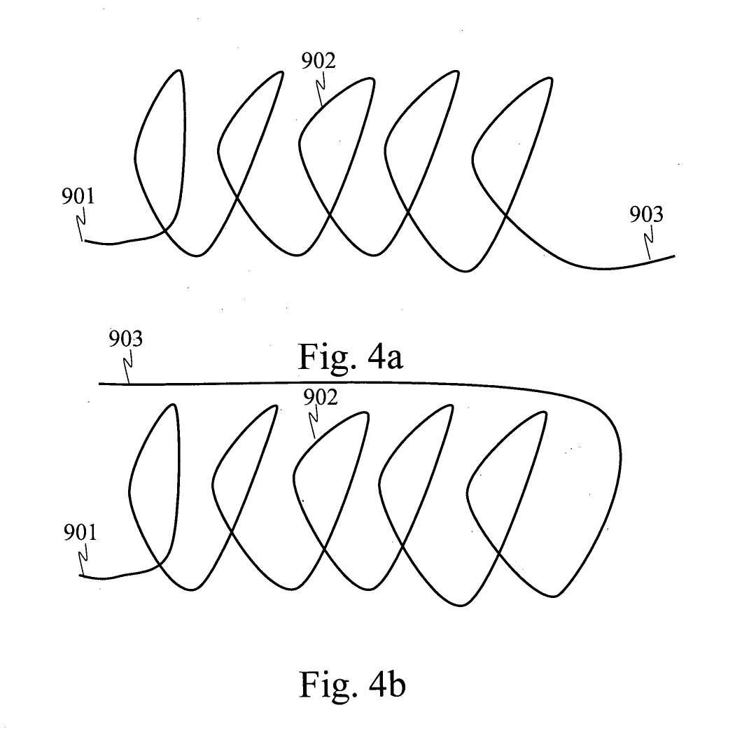

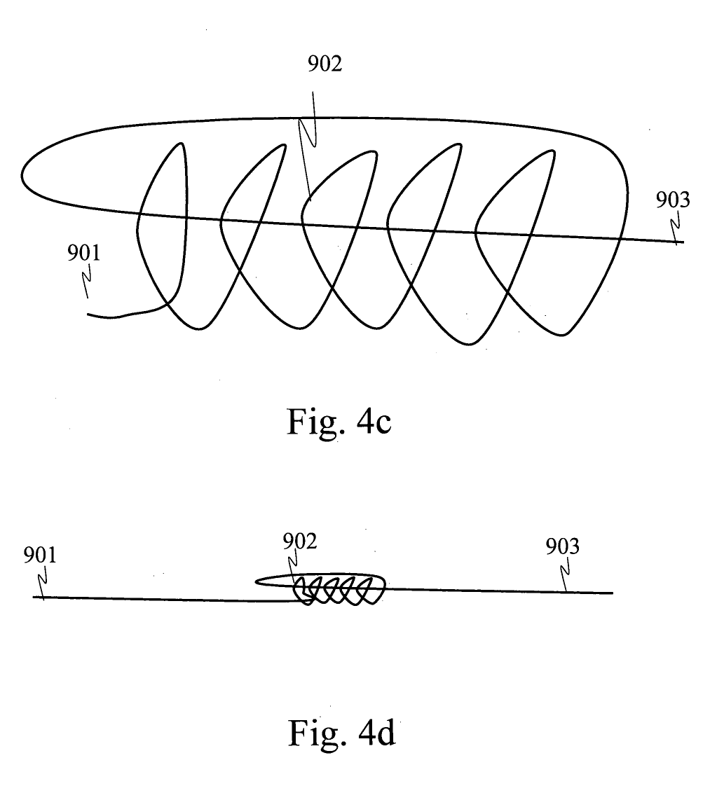

This embodiment is somehow a combination of the two main mbodiment of the present invention. On the one hand, it can be seen as a pre-coiled suture (FIG. 4) which comprises two kinds of sutures (31 and 35);

On the other hand, it can be seen as a suture coupled to a spring (or a flexible helical lumen), FIGS. 1a-1m, 2a-2b and 3a-3b.

According to another embodiment of the present invention, a method for internally forming a knot made of at least one loop in a suture within a body cavity is provided. Said knot is formed during surgeries, namely minimal invasive surgeries. The method comprises steps selected inter alia from:
a. obtaining a suturing device; the suture device comprises a flexible pre-shaped helical lumen having two opposite ends, said lumen comprising n loops; n is a real number higher than or equal to 1;
b. threading a first end of the suture through a first end of said pre-shaped helical lumen; thereby accommodating at least a portion of said suture within said helical lumen;
c. passing at least one end of the suture through y loops of said helical lumen from the opposite end of said lumen; y is an integer less than or equals to n; and,
d. pulling the passed end of the suture, thereby providing said knot.

According to another embodiment, the method as described above, additionally comprising step of repeating said step of passing at least one end of said suture through at least one loop of said helical lumen from the opposite end of said lumen k times, where k is an integer equal to, or higher than 1.

It is another object of the present invention to provide the method as defined above, additionally comprising step un threading said suture from said pre-shaped helical lumen by pulling out said pre-shaped helical lumen.

According to another embodiment, the method as described above, additionally comprising step of forming k knots.

According to another embodiment, the method as described above, additionally comprising step of selecting the materials from which said lumen is made of from a group consisting of shape memory materials, plastics, polymers, transparent materials, biocompatible materials, bioabsorbable materials, and drug-treated materials.

According to another embodiment, the method as described above, additionally comprising step of grasping and pulling a grabable portion such that said flexible pre-shaped helical lumen is transformed into a substantially straight lumen.

According to another embodiment, the method as described above, additionally comprising step of indicating proper threading of said at least one of said suture is obtained trough said lumen.

It is within provision of the invention that the suture itself be formed in the shape of a helix, such that a helical tube is not needed. The operations indicated in FIG. 4 are carried out as before, without the need to remove the helical tube from the area of the knot.

According to this embodiment, a suture for internally forming a knot in a suture within a body cavity, during surgeries (namely minimal invasive surgeries) is provided. The suture comprises at least one coiled section (helicoil) characterized by a predetermined number of loops. The coiled section is adapted for being threaded by one end of said suture, such that said knot is provided.

This embodiment is shown in FIGS. 4a-4d. In FIG. 4a the coil shaped section 902 is shown with ends 901 and 903:

In FIG. 4b the end 903 of the suture has been pulled towards the end 901.

In FIG. 4c the end 903 is pulled through the coil section 902 and out the other end. At this point a knot has been formed, topologically speaking. In FIG. 4d the ends 901, 903 have been pulled apart, tightening the knot.

According to another embodiment of the present invention, the suture as described above, is made of material selected from a group consisting of medical sutures, shape memory materials, nitinol, polymers, metals, transparent materials, biocompatible materials, bioabsorbable materials, non-bioabsorbable materials, mono/multi Filament and drug-treated materials.

The suture is formulated with it helical shape by means selected from extrusion, thermal treatment, plastic deformation, gluing et cetera.

According to another embodiment of the present invention, the diameter of said coiled section (helicoil) is in the range of about 1 mm to about 50 mm.

According to another embodiment of the present invention, the helicity (i.e., pitch) of said coiled section (helicoil) is in the range of about 0 mm to about 50 mm.

According to another embodiment of the present invention, the coiled section (HELICOIL) of the suture is characterized by a varying diameter and helicity.

It will be appreciated by one skilled in the art that if the coil has n loops and the suture is pulled through the coil k times, then k knots of n loops will be knotted onto the suture.

In essence one method of the instant invention consists of using a suturing device, said comprising a flexible pre-shaped helical tube. This helix has n loops. A suture is passed through the tube and thus takes the form of the helix.

This structure may now be passed inside the body of a patient undergoing laparoscopic surgery. To form a knot, one passes one end of the suture through the helix. It is important to passes the suture through the end of the helix that is more distant, as otherwise a knot will not form. We note in passing that the suture need not pass through the entire helix but can pass through an arbitrary number loops y of the helix. By pulling the suture tight, a knot of y loops is formed. As will be clear to one skilled in the art this process can be repeated in order to form k knots.

1. A suturing device for internally forming a knot in a suture within a body cavity, during surgeries, wherein said device comprising a flexible pre-shaped helical lumen having two opposite ends, said lumen comprising n loops; n is a real number higher than or equal to 1; said helical lumen is adapted to accommodate at least a portion of said suture, such that said knot is obtained by threading by at least one end of said suture through at least one of said loops.

2. The device of claim 1, wherein said lumen is made of materials selected from a group consisting of shape memory materials, polymers, metals, plastics, transparent materials, biocompatible materials, bioabsorbable materials, and drug-treated materials.

3. The device of claim 1, wherein said device additionally comprising a grabable portion adapted to be pulled such that said flexible pre-shaped helical lumen is transformed into a substantially straight lumen.

4. The device of claim 1, additionally comprising at least one drag coupled to one end of said lumen so as to distinguish between the two ends of the same.

5. The device of claim 1, wherein one end of said lumen additionally comprises indicating means adapted to indicate a proper threading of said at least one of said suture is obtained.

6. The device of claim 1, wherein said lumen comprises at least one groove and/or at least one opening on the outer surface of the same; said groove and/or said opening is adapted to facilitate threading of said suture through said lumen.

7. The device of claim 1, wherein the cross sectional area of said lumen is selected from a group consisting of circular, polynomial rectangular or any combination thereof.

8. A method for internally forming a knot made of at least one loop in a suture within a body cavity, during surgeries, said method comprising steps of:
a. obtaining a suturing device, said suture device comprising a flexible pre-shaped helical lumen having two opposite ends, said lumen comprising n loops; n is real number higher than or equal to 1;
b. threading a first end of said suture through said first end of said pre-shaped helical lumen; thereby accommodating at least a portion of said suture within said helical lumen;
c. passing at least one end of said suture through y loops of said helical lumen from the opposite end of said lumen; y is an integer less than or equals to n; and, d. pulling said passed end of said suture, thereby providing said knot.

9. The method according to claim 8, additionally comprising step un threading said suture from said pre-shaped helical lumen by pulling out said pre-shaped helical lumen.

10. The method according to claim 8, additionally comprising step of repeating said step of passing at least one end of said suture through at least one loop of said helical lumen from the opposite end of said lumen k times, where k is an integer higher than 1.

11. The method according to claim 8, additionally comprising step of forming k knots.

12. The method according to claim 8, additionally comprising step of selecting the materials from which said lumen is made of from a group consisting of shape memory materials, polymers, metals, transparent materials, biocompatible materials, bioabsorbable materials, and drug-treated materials.

13. The method according to claim 8, additionally comprising step of grasping and pulling a grabable portion such that said flexible pre-shaped helical lumen is transformed into a substantially straight lumen.

14. The method according to claim 8, additionally comprising step of forming said lumen with at least one drag coupled to one end of said lumen so as to distinguish between the two ends of the same.

15. The method according to claim 8, additionally comprising step of providing said lumen with at least one groove or/and at least one opening on the outer surface of the same for facilitating said step of threading of said suture through said lumen.

16. The method according to claim 8, additionally comprising step of selecting the cross sectional area of said lumen from a group consisting of circular, polynomial, polygon, rectangular or any combination thereof.

17. The method according to claim 8, additionally comprising step of indicating proper threading of said at least one of said suture is obtained trough said loops.

18. A suture for internally forming a knot in a suture within a body cavity, during surgeries, wherein said suture comprises at least one coiled section (helicoil) characterized by a predetermined number of loops; said coiled section (helicoil) adapted for being threaded by one end of said suture, such that said knot is provided.

19. The suture of claim 18, wherein said at least one coiled section (helicoil) is provided by means selected from extrusion, thermal treatment, plastic deformation, gluing or any combination thereof

20. The suture of claim 18, wherein said suture is made of material selected from a group consisting of medical sutures, shape memory materials, nitinol, polymers, metals, transparent materials, biocompatible materials, bioabsorbable materials, non-bioabsorbable materials, mono/multi Filament and drug-treated materials.

21. The suture of claim 18, wherein the diameter of said coiled section (helicoil) is in the range of about 1 mm to about 50 mm.

22. The suture of claim 18, wherein the helicity of said coiled section (helicoil) is in the range of about 0 mm to about 50 mm.

23. The suture of claim 18, wherein said coiled section (helicoil) is characterized by a varying diameter and helicity.

24. The suture of claim 18, additionally comprising a second suture; wherein said second suture is characterized by at least one selected from a group consisting of mechanical properties, chemical properties or any combination thereof which are substantially different than said suture.

25. The suture of claim 24, additionally comprising coupling means 32 adapted to couple said suture and said second suture; wherein said coupling is provided via mechanical means, chemical means, physical means or any combination thereof

26. The suture of claim 24, wherein said coupling is provided by at least one selected from a group consisting of hooks and loops, cramping, ratchet, glue or any combination thereof

27. The suture of claim 24, additionally comprising balancing element 33 adapted to balance said suture.

28. A method for internally forming a knot made of at least one loop in a suture within a body cavity, during surgeries, said method comprising steps of:
a. providing a suture having at least one coiled section (helicoil); said coiled section (helicoil) comprises preset n loops; n is a real number higher or equal to 1;
b. passing at least one end of said suture through y loop of said coiled; y is an integer less than or equals to n; and,
c. pulling said passed end of said suture, thereby forming a knot therein.

29. The method of claim 28, additionally comprising step of forming said suture from material selected from a group consisting of medical sutures, shape memory materials, nitinol, polymers, metals, transparent materials, biocompatible materials, bioabsorbable materials, non-bioabsorbable materials, mono/multi Filament and drug-treated materials.

30. The method of claim 28, additionally comprising step of providing the diameter of said coiled section (helicoil) is in the range of about 1 mm to about 50 mm.

31. The method of claim 28, additionally comprising step of forming said at least one coiled section (helicoil) by means selected from extrusion, thermal treatment, plastic deformation, gluing or any combination thereof.

32. The method of claim 28, additionally comprising step of the helicity (i.e., pitch) of said coiled section (helicoil) is in the range of about 0 mm to about 50 mm.

33. The method of claim 28, additionally comprising step of providing a second suture; wherein said second suture is characterized by at least one selected from a group consisting of mechanical properties, chemical properties or any combination thereof which are substantially different than said suture.

34. The method of claim 28, additionally comprising step of coupling said suture and said second suture.

35. The method of claim 34, wherein comprising step of coupling is provided via mechanical means, chemical means, physical means or any combination thereof.

36. The method of claim 34, wherein comprising step of coupling is provided by at least one selected from a group consisting of hooks and loops, cramping, ratchet, glue or any combination thereof
